(19) United States
(12) Patent Application Publication
(10) Pub. No.: US 2025/0177312 A1
(43) Pub. Date: Jun. 5, 2025

(54) MODIFIED RELEASE VALPROIC ACID SOFTGEL CAPSULE

(71) Applicant: R.P. SCHERER TECHNOLOGIES, LLC, Carson City, NV (US)

(72) Inventors: Karunakar Sukuru, St. Petersburg, FL (US); Qi Fang, St. Petersburg, FL (US)

(21) Appl. No.: 18/838,825

(22) PCT Filed: Feb. 17, 2023
(86) PCT No.: PCT/US2023/013277

§ 371 Date: Aug. 15, 2024

Related U.S. Application Data
(60) Provisional application No. 63/311,559, filed on Feb. 18, 2022.

(51) Int. Cl. A61K 9/48; A61K 31/19

(52) U.S. Cl. CPC A61K 9/4825; A61K 9/4833; A61K 9/4858; A61K 31/19

(57) ABSTRACT

Softgel capsules comprise a fill composition and a shell composition, wherein the fill composition includes valproic acid and the shell composition includes gelatin, a plasticizer, and an enteric polymer. The softgel capsules further include from 125 mg to 1000 mg dosing of valproic acid. A method for producing the softgel capsule using a gel mass and rotary die encapsulation is also provided.

### CROSS REFERENCE TO RELATED APPLICATION(S)

The present application claims priority to U.S. Provisional Patent Application No. 63/311,559 filed on Feb. 18, 2022. The entire contents of which is incorporated in its entirety.

### FIELD OF THE INVENTION

The present invention relates to an enteric softgel capsule, wherein the softgel capsule includes a shell composition and a fill composition. Specifically, the enteric softgel capsule includes valproic acid in the fill composition. Methods of preparation of such dosage forms and methods of use thereof are also disclosed.

### BACKGROUND OF THE INVENTION

Capsule dosage forms are commonly used for oral administration of a variety of pharmaceuticals. The capsules can be, for example, soft gelatin shell or hard shell (animal or vegetable variety). Softgel capsules provide numerous advantages including fast dissolution, taste-masking, ease of swallowing, fewer excipients as compared to tablets, delivery of a liquid matrix that solubilizes and improves oral bioavailability of a marginally hydrophilic compound, delivery of low and ultra-low doses of a compound, delivery of low melting temperature compounds and minimization of dust generation during manufacturing and thus, improved safety for production personnel.

Soft capsules, in particular, soft gelatin capsules (or softgel capsules), provide a dosage form which is more readily accepted by patients, since the capsules are easy to swallow and need not be flavored in order to mask any unpleasant taste of the active agent. Softgel encapsulation of drugs further provides the potential to improve the bioavailability of the pharmaceutical agents. For example, active ingredients may be rapidly released in liquid or solution form as soon as the gelatin shell ruptures.

### SUMMARY OF THE INVENTION

The present invention advances the state of the art by developing delayed release oral dosage forms, in particular, delayed release softgel capsules, that include an enteric polymer. The delayed release softgel capsules of the present invention are related to a fill material and a shell composition, wherein the fill material includes valproic acid.

In an embodiment of the present disclosure, a softgel capsule includes a fill material and a shell composition, wherein the fill material comprises valproic acid, and wherein the shell composition comprises a gelatin, a plasticizer, and an enteric polymer.

In some embodiments of the softgel capsule, wherein the shell composition further includes dextrose. In some embodiments of the softgel capsule, wherein the enteric polymer may be pectin. In certain embodiments, the pectin may be an amidated pectin, a non-amidated pectin or combinations thereof.

In some embodiments of the softgel capsule, the shell composition may include about 25 wt % to about 55 wt % of a gelatin. In some embodiments of the softgel capsule, wherein the shell composition may include about 3 wt % to about 22 wt % of pectin. In some embodiments of the softgel capsule, the shell composition may include about 0.01 wt % to about 1.0 wt % of dextrose.

In certain embodiments of the softgel capsule, the gelatin may be selected from the group consisting of Type A gelatin, Type B gelatin and mixtures thereof. In certain embodiments of the softgel capsule, the gelatin may be selected from the group consisting of fish gelatin, hide gelatin, bone gelatin and mixtures thereof.

In some embodiments of the softgel capsule, the plasticizer may be selected from the group consisting of glycerin, aqueous sorbitol and sorbitan solution and combinations thereof. In certain embodiments, the plasticizer may be glycerin, aqueous sorbitol and sorbitan solution or combinations thereof.

In some embodiments of the softgel capsule, the shell composition may further include water. In certain embodiments of the softgel capsule, the shell composition may include from about 10 wt % to about 50 wt % of water.

In some embodiments of the softgel capsule, the shell composition may further include a gelling agent. In certain embodiments, the shell composition may include from about 0.2 wt % to about 8 wt % of the gelling agent. In certain embodiments, the gelling agent may be a gellan gum.

In some embodiments, the fill composition may include an antioxidant. In some embodiments, the film composition may include butylated hydroxytouene (BHT). In some embodiments, the fill composition may include butylated hydroxyanisole (BHA).

In some embodiments of the softgel capsule, the fill composition may include from about 125 mg to about 1000 mg of valproic acid, from about 250 mg to about 750 mg, or from about 350 mg to about 550 mg, or any sub ranges or values within.

In some embodiments of the softgel capsule, the shell composition may include non-amidated pectin. In certain embodiments of the softgel capsule, the shell composition may further include a methacrylic copolymer.

In some embodiments of the softgel capsule, the softgel capsule may have a stability of at least about 90% when tested at 25° C./60% RH at 1 month, 2 months, 3 months, 6 months, 12 months, or 24 months. In certain embodiments, the softgel capsule may have a stability of at least about 92% when tested at 25° C./60% RH at 1 month, 2 months, 3 months, 6 months, 12 months, or 24 months. In certain embodiments, the softgel capsule may have a stability of at least about 94% when tested at 25° C./60% RH at 1 month, 2 months, 3 months, 6 months, 12 months, or 24 months. In certain embodiments, the softgel capsule may have a stability of at least about 96% when tested at 25° C./60% RH at 1 month, 2 months, 3 months, 6 months, 12 months, or 24 months. In certain embodiments, the softgel capsule may have a stability of at least about 98% when tested at 25° C./60% RH at 1 month, 2 months, 3 months, 6 months, 12 months, or 24 months. In certain embodiments, the softgel capsule may have a stability of at least about 99% when tested at 25° C./60% RH at 1 month, 2 months, 3 months, 6 months, 12 months, or 24 months.

In some embodiments, the softgel capsule may have a stability of at least about 90% when tested at 30° C./65% RH at 1 month, 2 months, 3 months, 6 months, or 12 months. In certain embodiments, the softgel capsule may have a stability of at least about 92% when tested at 30° C./65% RH at 1 month, 2 months, 3 months, 6 months, or 12 months. In certain embodiments, the softgel capsule may have a stability of at least about 94% when tested at 30° C./65% RH at 1 month, 2 months, 3 months, 6 months, or 12 months. In certain embodiments, the softgel capsule may have a stability of at least about 96% when tested at 30° C./65% RH at 1 month, 2 months, 3 months, 6 months, or 12 months. In certain embodiments, the softgel capsule may have a stability of at least about 98% when tested at 30° C./65% RH at 1 month, 2 months, 3 months, 6 months, or 12 months. In some embodiments, the softgel capsule may have a stability of at least about 99% when tested at 30° C./65% RH at 1 month, 2 months, 3 months, 6 months, or 12 months.

In some embodiments, the softgel capsule may have a stability of at least about 90% when tested at 40° C./75% RH at 1 month, 2 months, 3 months, or 6 months. In certain embodiments, the softgel capsule may have a stability of at least about 92% when tested at 40° C./75% RH at 1 month, 2 months, 3 months, or 6 months. In certain embodiments, the softgel capsule may have a stability of at least about 94% when tested at 40° C./75% RH at 1 month, 2 months, 3 months, or 6 months. In certain embodiments, the softgel capsule may have a stability of at least about 96% when tested at 40° C./75% RH at 1 month, 2 months, 3 months, or 6 months. In certain embodiments, the softgel capsule may have a stability of at least about 98% when tested at 40° C./75% RH at 1 month, 2 months, 3 months, or 6 months. In certain embodiments, the softgel capsule may have a stability of at least about 99% when tested at 40° C./75% RH at 1 month, 2 months, 3 months, or 6 months.

In some embodiments, the softgel capsule may remain intact for at least about 15 minutes, at least about 30 minutes, at least about 1 hour, at least about 2 hours, at least about 3 hours, at least about 4 hours, or at least about 5 hours in an acidic medium, wherein a dissolution test is performed in a USP Apparatus II with paddles at a speed of 50 rpm in pH 1.2 medium.

In some embodiments, the softgel capsule may disintegrate in pH 6.8 buffer in about 60 minutes or less, in about 45 minutes or less, in about 30 minutes or less, in about 20 minutes or less, in about 10 minutes or less, or in about 5 minutes or less.

In some embodiments, the softgel capsule may remain intact for at least about 15 minutes, at least about 30 minutes, at least about 1 hour, at least about 2 hours, at least about 3 hours, at least about 4 hours, or at least about 5 hours in pH 1.2 medium, dissolution test is performed in a USP Apparatus II with paddles at a speed of 50 rpm in pH 1.2 medium, and may disintegrate in pH 6.8 buffer in about 60 minutes or less, in about 45 minutes or less, in about 30 minutes or less, in about 20 minutes or less, in about 10 minutes or less, or in about 5 minutes or less.

In some embodiments of the softgel capsule, the total impurity may not exceed about 0.5%, about 0.4%, about 0.3%, about 0.2%, about 0.1% or about 0.01%, wherein the total impurity is measured per current USP Monograph test method. In certain embodiments of the softgel capsule, the total impurity does not exceed about 0.3%.

In some embodiments of the present disclosure, a process for producing the softgel capsule of the present disclosure includes encapsulating the fill composition in the shell composition to form the softgel capsule and drying the softgel capsule. In some embodiments of the process, prior to encapsulating a gel conversion occurs for the shell composition.

In some embodiments, a method of treating seizures may include administering a softgel capsule of the present disclosure. In certain embodiments, a method of treating bipolar disorder may include administering a softgel capsule of the present disclosure. In certain embodiments, a method of treating migraines may include administering a softgel capsule of the present disclosure.

### DETAILED DESCRIPTION OF THE INVENTION

The present invention advances the state of the art by developing modified or delayed release oral dosage forms, in particular, delayed release softgel capsules, that include an enteric polymer. The delayed release softgel capsules of the present invention do not dissolve in a gastric environment of the stomach, but rather dissolve in the intestines. Such mechanism is beneficial for delivery of active ingredients that may cause stomach irritation or degradation in the acidic environment of the stomach.

As used herein, “pharmaceutically active ingredient” refers to a drug or compound that may be used in the diagnosis, cure, mitigation, treatment, or prevention of a condition. The term “condition” or “conditions” refers to those medical conditions that can be treated or prevented by administration to a subject of an effective amount of an active agent.

As used herein, the term “active ingredient” refers to any material that is intended to produce a therapeutic, prophylactic, or other intended effect, whether or not approved by a government agency for that purpose. This term with respect to a specific agent includes the pharmaceutically active agent, and all pharmaceutically acceptable salts, solvates and crystalline forms thereof, where the salts, solvates and crystalline forms are pharmaceutically active.

As used herein, the terms “therapeutically effective” and an “effective amount” refer to the amount of active agent or the rate at which it is administered which is needed to produce a desired therapeutic result.

As used herein, “shell” or “shell composition” refers to the shell of a softgel capsule which encapsulates a fill material.

All references to wt % throughout the specifications and the claims refer to the weight of the component in reference to the weight of the entire composition and may also be designated as w/w.

As used herein, “fill material” or “fill” refers to the composition that is encapsulated by the capsule shell and contains at least one pharmaceutically active ingredient.

As used herein, “about” refers to any values that are within a variation of +10%, such that “about 10” would include from 9 to 11. As used herein, “a,” “an,” or “the” refers to one or more, unless otherwise specified. Thus, for example, reference to “an excipient” includes a single excipient as well as a mixture of two or more different excipients, and the like.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

In some embodiments, the active pharmaceutical ingredient may be valproic acid or salts thereof. In some embodiments, the active pharmaceutical ingredient may include valproate, sodium valproate, or valproate semisodium.

According to an embodiment, a softgel capsule may include a fill composition and a shell composition, wherein the fill composition may include a pharmaceutically active ingredient, wherein the shell composition includes a gelatin, a plasticizer, and an enteric polymer. In some embodiments, the enteric polymer may be an amidated pectin, non-amidated pectin or a combination thereof. In some embodiments, the plasticizer may be glycerin.

In some embodiments, the fill composition may include a pharmaceutically active ingredient. In other embodiments, the fill composition may include additional fill components such as flavoring agents, sweetening agents, coloring agents and fillers, an antioxidant or other pharmaceutically acceptable excipients or additives such as synthetic dyes and mineral oxides.

In some embodiments, the antioxidant may be butylated hydroxytoluene (BHT), butylated hydroxyanisole (BHA), alfa tocopherol, vitamin E TPGS, propyl gallate, cysteine, sodium metabisulfite, or a combination thereof.

In some embodiments, the fill composition may include the pharmaceutically active ingredient in an amount of about 125 mg, about 250 mg, about 500 mg, about 600 mg, about 700 mg, about 800 mg, about 900 mg, about 1000 mg, about 1100 mg, about 1200 mg, or about 1300 mg. In some embodiments, the fill composition may include the pharmaceutically active ingredient in an amount from about 125 mg to about 1000 mg, from about 250 mg to about 750 mg, or from about 350 mg to about 550 mg, or any sub ranges or values within.

In some embodiments, an antioxidant may be included in the fill composition. The antioxidant may be included in an amount of about 0.001 parts by weight to about 2 parts by weight based on 100 parts by weight based on the fill composition.

In an embodiment, the gelatin in the shell composition may include Type A gelatin, Type B gelatin, a hide or skin gelatin and/or a bone gelatin used alone or in combination. In one embodiment, the gelatin may be a pigskin gelatin or a Type B. In another embodiment, there may be only one type of gelatin. In yet another embodiment, the gelatin may be a combination of at least two types of gelatins. In an embodiment, the amount of gelatin in the shell composition may be about 20 wt % to about 80 wt %, or from about 30 wt % to about 60 wt %, or from about 25 wt % to about 55 wt %.

In one embodiment, the shell composition includes dextrose. In an embodiment, the amount of dextrose in the shell composition is about 0.005 wt % to about 5 wt %, or about 0.01 wt % to about 4 wt %, or about 0.01 wt % to about 3 wt %, or about 0.01 wt % to about 2 wt %, or about 0.01 wt % to about 1 wt %, or from about 0.1 wt % to about 3 wt %, or from about 0.1 wt % to about 2 wt %, or from about 0.1 wt % to about 1 wt %, or from about 0.15 wt % to about 2 wt %, or from about 0.15 to about 1 wt %. The dextrose may be added to the capsule shell to mitigate potential reduction in gel strength. The concentration of dextrose in the shell composition may be in an effective amount to improve the enteric property but not so high that it would interfere with the rupture of the softgel capsule.

In some embodiments, the shell composition may include pectin. In some embodiments, the pectin may be a low methoxy pectin. In some embodiments, the pectin may be an amidated pectin, non-amidated pectin or a combination thereof. In an embodiment, the pectin is low methyl (LM) pectin with Degree of Esterification lower than 50. In some embodiments, the pectin is LMS-318, SPL-12, LM-102 AS-Z and/or LM-12 CG-Z. In other embodiments, the low methoxy (LM) pectin may be LM Pectin (P-25), LM Pectin (445C), LM Pectin (100C) or a combination thereof. Too much pectin in the dosage form may reduce the gel strength of the softgel capsule which may in turn adversely affect the sealability of the softgel capsule. Therefore, pectin may be added to the dosage form at a concentration that is sufficiently high to form a delayed release dosage form and at the same time is sufficiently low to mitigate the reduction in gel strength. In an embodiment, an amount of pectin in the shell composition is from about 2 wt % to about 25 wt %, from about 3 wt % to about 25 wt %, from about 4 wt % to about 25 wt %, or from about 5 wt % to about 25 wt %, or from about 5 wt % to about 20 wt %, or from about 5 wt % to about 15 wt %, or from about 5 wt % to about 10 wt %.

In an embodiment, the shell composition may also include a plasticizer. In some embodiments, the plasticizer in the shell composition may include glycerin, aqueous sorbitol and sorbitan solution and combinations thereof. Other suitable plasticizers may include, but not be limited to, sugar alcohol plasticizer such as isomalt, maltitol, xylitol, erythritol, adonitol, dulcitol, pentaerythritol, or mannitol; or polyol plasticizer such as diglycerin, dipropylene glycol, a polyethylene glycol up to 10,000 MW, neopentyl glycol, propylene glycol, 1,3-propanediol, 2-methyl-1,3-propanediol, trimethylolpropane, a polyether polyol, ethanol amines; and mixtures thereof. Other exemplary plasticizers may also include, without limitations, low molecular weight polymers, oligomers, copolymers, oils, small organic molecules, low molecular weight polyols having aliphatic hydroxyls, ester-type plasticizers, glycol ethers, poly (propylene glycol), multi-block polymers, single block polymers, citrate ester-type plasticizers, and triacetin. Such plasticizers may include 1,2-butylene glycol, 2,3-butylene glycol, styrene glycol, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, triethyl citrate, glyceryl monostearate, polysorbate 80, acetyl triethyl citrate, tributyl citrate and allyl glycolate, and mixtures thereof.

In an embodiment, the amount of plasticizer in the shell composition is about 2 wt % to about 40 wt %, or from about 5 wt % to about 35 wt %, or from about 10 wt % to about 30 wt %, or from about 15 wt % to about 25 wt %.

In an embodiment, the shell composition may also include a gelling agent. In some embodiments, the gelling agent may be a gellan gum, agar, alginate, guar gum or Locus bean gum. For example, the gellan gum may be a low acyl gellan gum, for example Kelcogel CG-LA.

The shell composition may also include water. In some embodiments, water may be included in the shell composition in an amount of 5 wt % to about 30 wt %, or from about 10 wt % to about 25 wt %, or about 15 wt % to about 20 wt % based on the total shell composition.

In an embodiment, the shell composition may optionally comprise additional agents such as coloring agents, flavorings agents, sweetening agents, fillers, antioxidants, diluents, pH modifiers or other pharmaceutically acceptable excipients or additives such as synthetic dyes and mineral oxides.

Exemplary suitable coloring agents may include, but not be limited to, colors such as e.g., white, black, yellow, blue, green, pink, red, orange, violet, indigo, and brown. In specific embodiments, the color of the dosage form can indicate the contents (e.g., one or more active ingredients) contained therein.

Exemplary suitable flavoring agents may include, but not be limited to, “flavor extract” obtained by extracting a part of a raw material, e.g., animal or plant material, often by using a solvent such as ethanol or water; natural essences obtained by extracting essential oils from the blossoms, fruit, roots, etc., or from the whole plants.

Additional exemplary flavoring agents that may be in the dosage form may include, but not be limited to, breath freshening compounds like menthol, spearmint, and cinnamon, coffee beans, other flavors or fragrances such as fruit flavors (e.g., cherry, orange, grape, etc.), especially those used for oral hygiene, as well as actives used in dental and oral cleansing such as quaternary ammonium bases. The effect of flavors may be enhanced using flavor enhancers like tartaric acid, citric acid, vanillin, or the like.

Exemplary sweetening agents may include, but not be limited to, one or more artificial sweeteners, one or more natural sweeteners, or a combination thereof. Artificial sweeteners include, e.g., acesulfame and its various salts such as the potassium salt (available as Sunett®), alitame, aspartame (available as NutraSweet® and Equal®), salt of aspartame-acesulfame (available as Twinsweet®), neohesperidin dihydrochalcone, naringin dihydrochalcone, dihydrochalcone compounds, neotame, sodium cyclamate, saccharin and its various salts such as the sodium salt (available as Sweet'N Low®), stevia, chloro derivatives of sucrose such as sucralose (available as Kaltame® and Splenda®), and mogrosides. Natural sweeteners include, e.g., glucose, dextrose, invert sugar, fructose, sucrose, glycyrrhizin; monoammonium glycyrrhizinate (sold under the trade name MagnaSweet®); Stevia rebaudiana (Stevioside), natural intensive sweeteners, such as Lo Han Kuo, polyols such as sorbitol, mannitol, xylitol, erythritol, and the like.

In some embodiments, a methacrylic copolymer may be included in the shell composition. In an embodiment, the shell composition may include a methacrylic copolymer in an amount of about 0.1 wt % to about 5 wt %, or from about 1 wt % to about 4 wt %, or from about 2 wt % to about 3 wt % based on the total weight of the shell composition. In some embodiments, the methacrylic copolymer may be Kollicoat MAE 100P. Without being limited to a theory, the inventors believe that the methacrylic copolymer enhances the enteric property of the shell composition.

In some embodiments, the softgel capsule may provide a delayed release or modified release of the active agent.

In some embodiments, the shell composition and/or the softgel capsule may be tested in a dissolution test. In one embodiment, the dissolution test is a two-stage dissolution. The dissolution may be performed in a USP Apparatus II with paddles at a speed of 50 rpm to 100 rpm in pH 1.2 medium, (with or without pepsin) or gastric fluid. The softgel capsule according to this embodiment may remain intact for at least about 15 minutes, at least about 30 minutes, at least about one hour, at least about two hours, at least about three hours, at least about four hours, or at least about five hours, in acidic medium and may disintegrate in pH 6.8 buffer or intestinal fluid in about 60 minutes or less, in about 45 minutes or less, in about 30 minutes or less, in about 20 minutes or less, in about 10 minutes or less, or in about 5 minutes or less. The acidic medium may be 0.1N HCl.

In some embodiments, the shell composition and/or the softgel capsule may be tested in a disintegration test performed in a USP/EP Disintegration Apparatus in pH 1.2 medium (with or without pepsin) or simulated gastric fluid. The softgel capsule according to this embodiment may remain intact for at least about 15 minutes, at least about 30 minutes, at least about one hour, at least about two hours, at least about three hours, at least about four hours, or at least about five hours, in acidic medium and may disintegrate in pH 6.8 buffer or intestinal fluid in about 60 minutes or less, in about 45 minutes, in about 30 minutes or less, in about 20 minutes or less, in about 10 minutes or less, or in about 5 minutes or less.

In some embodiments, the dissolution test may be performed for about 150 minutes, about 120 minutes, about 105 minutes, about 90 minutes, about 75 minutes, about 60 minutes, about 45 minutes, about 30 minutes, about 15 minutes, about 10 minutes, or about 5 minutes.

In some embodiments, the softgel capsule may be ethanol resistant. Resistance to ethanol is measured using the USP dissolution apparatus II with a medium that contains 10%, 20%, 30%, 40% or 50% ethanol. The softgel capsule may stay intact for about 150 minutes, about 120 minutes, about 105 minutes, about 90 minutes, about 75 minutes, about 60 minutes, about 45 minutes, about 30 minutes, or about 15 minutes.

In some embodiments, the softgel capsule may have a stability of at least about 90%, at least about 91%, at least about 92%, at least about 93%, at least about 94%, at least about 95%, at least 96%, at least 97%, at least about 98% or at least 99% when tested at 25° C./60% RH at 1 month, 2 months, 3 months, 6 months, 12 months, or 24 months. In some embodiments, the softgel capsule may have a stability from about 90% to about 110%, from about 92% to about 108%, from about 94% to about 106%, from about 96% to about 104%, from about 98% to about 102% or from about 99% to about 101% when tested at 25° C./60% RH at 1 month, 2 months, 3 months, 6 months, 12 months or 24 months. In some embodiments, the softgel capsule may have a stability of at least about 90%, at least about 91%, at least about 92%, at least about 93%, at least about 94%, at least about 95%, at least 96%, at least 97%, at least about 98% or at least 99% when tested at 30° C./65% RH at 1 month, 2 months, 3 months, 6 months or 12 months. In some embodiments, the softgel capsule may have a stability from about 90% to about 110%, from about 92% to about 108%, from about 94% to about 106%, from about 96% to about 104%, from about 98% to about 102% or from about 99% to about 101% when tested at 30° C./65% RH at 1 month, 2 months, 3 months, 6 months, or 12 months. In some embodiments, the softgel capsule may have a stability of at least about 90%, at least about 91%, at least about 92%, at least about 93%, at least about 94%, at least about 95%, at least 96%, at least 97%, at least about 98% or at least 99% when tested at 40° C./75% RH at 1 month, 2 months, 3 months, or 6 months. In some embodiments, the softgel capsule may have a stability from about 90% to about 110%, from about 92% to about 108%, from about 94% to about 106%, from about 96% to about 104%, from about 98% to about 102% or from about 99% to about 101% when tested at 40° C./75% RH at 1 month, 2 months, 3 months, or 6 months. As used herein, stability is measured as the amount of valproic acid remaining after an accelerated time period stored at a specified temperature and relative humidity.

In certain embodiments, any of the compositions described herein may exhibit any of the above described chemical stability upon storage for an extended duration (e.g., one week, two weeks, three weeks, one month, two months, three months, four months, five months, six months, seven months, eight months, nine months, ten months, eleven months, twelve months, fifteen months, eighteen months, twenty one months, or twenty four months, or any sub-range or single value therein) at a temperature ranging from about 5° C. to about 50° C., from about 10° C. to about 40° C., from about 20° C. to about 30° C., or any sub-range or single value therein, at a relative humidity ranging from about 20% to about 75%.

In certain embodiments, any of the compositions described herein may exhibit physical stability upon storage for an extended duration (e.g., one week, two weeks, three weeks, one month, two months, three months, four months, five months, six months, seven months, eight months, nine months, ten months, eleven months, twelve months, fifteen months, eighteen months, twenty one months, or twenty four months, or any sub-range or single value therein) at a temperature ranging from about 5° C. to about 50° C., from about 10° C. to about 40° C., from about 20° C. to about 30° C., or any sub-range or single value therein, at a relative humidity ranging from about 20% to about 75%.

In certain embodiments, the physical stability of the delayed release softgel capsules may be evidenced by the dissolution profile of the capsule in acidic medium and in buffer medium. For instance, the dissolution profile of the capsule in acidic medium and in buffer medium is substantially similar (or within specifications), after storage for up to 12 months, up to 6 months, up to 3 months, or up to 1 months (at ambient conditions or at stressed conditions of 40° C. and 75% relative humidity for any of these durations) as compared to the dissolution profile of the capsule before storage.

The term “substantially similar” may refer to a particular value being within about 30%, within about 25%, within about 20%, within about 15%, within about 10%, within about 5%, or within about 1% of a corresponding comparative value. The percentage being calculated based on the face value of the comparative value. For instance, a dissolution time range of 27 minutes to 33 minutes may be considered within 10% of comparative dissolution time of 30 minutes.

In some embodiments, the amount of impurity RRT 0.152 in the softgel is at most about 0.05%, at most about 0.04%, at most about 0.03%, at most about 0.02%, or at most about 0.01%, or from about 0.001% to about 0.05%, from about 0.01% to about 0.04%, or from about 0.02% to about 0.03%, any sub-range or single value therein. In some embodiments, the amount of impurity, butyric acid, in the softgel is at most about 0.05%, at most about 0.04%, at most about 0.03%, at most about 0.02%, or at most about 0.01%, or from about 0.001% to about 0.05%, from about 0.01% to about 0.04%, or from about 0.02% to about 0.03%, or any sub-range or single value therein. In some embodiments, the amount of impurity, valeric acid, in the softgel is at most about 0.05%, at most about 0.04%, at most about 0.03%, at most about 0.02%, or at most about 0.01%, or from about 0.001% to about 0.05%, from about 0.01% to about 0.04%, or from about 0.02% to about 0.03%, or any sub-range or single value therein. In some embodiments, the amount of impurity RRT 0.545 in the softgel is at most about 0.05%, at most about 0.04%, at most about 0.03%, at most about 0.02%, or at most about 0.01%, or from about 0.001% to about 0.05%, from about 0.01% to about 0.04%, or from about 0.02% to about 0.03%, or any sub-range or single value therein. In some embodiments, the amount of impurity RRT 0.873 in the softgel is at most about 0.05%, at most about 0.04%, at most about 0.03%, at most about 0.02%, or at most about 0.01%, or from about 0.001% to about 0.05%, from about 0.01% to about 0.04%, or from about 0.02% to about 0.03%, or any sub-range or single value therein. In some embodiments, the amount of impurity RRT 1.397 in the softgel is at most about 0.05%, at most about 0.04%, at most about 0.03%, at most about 0.02%, or at most about 0.01%, or from about 0.001% to about 0.05%, from about 0.01% to about 0.04%, or from about 0.02% to about 0.03%, or any sub-range or single value therein.

In some embodiments, the total impurities present in the softgel capsule does not exceed about 0.01%, about 0.05%, about 0.1%, about 0.15%, about 0.2%, about 0.25%, about 0.3%, about 0.35%, about 0.4%, about 0.45%, about 0.5%, or any sub-range or single value therein. In some embodiments, the total impurities present in the softgel capsule may be in the amount from about 0.0001% to about 0.5%, from about 0.001% to about 0.4%, from about 0.01% to about 0.3%, or from about 0.1% to about 0.2%, or any sub-range or single value therein.

Encapsulation of the fill material can be accomplished in any conventional manner. As an example, a rotary die encapsulation may be used. In some embodiments, gel conversion may be used to encapsulate the fill composition. In some embodiments, the gel conversion includes the shell composition, an opacifier and a colorant. The opacifier may be titanium dioxide. The colorant may include FD&C Yellow #6. The gel conversion may also include water. In some embodiments, the colorant may be included in an amount of about 0.5 g to about 2.5 g per kilogram of the shell composition. In some embodiments, the opacifier may be included in an amount of about 2 g to about 10 g per kilogram of the shell composition. In other embodiments, water may be included in an amount of about 40 grams to about 60 grams per kilogram of the shell composition.

According to an embodiment, the softgel capsule is prepared by (a) preparing the fill composition, the fill composition including a pharmaceutically active ingredient; and (b) encapsulating the fill composition in a shell composition. The encapsulation process may also include a sub-step of preparing the shell composition by, for example, admixing a gelatin, dextrose, a pectin and optionally a plasticizer. The shell composition may further undergo a gel conversion before encapsulation by adding colorants and water.

The softgel capsule of the present disclosure may be packaged in a blister pack, bottle or a volume pack.

### Examples

Specific embodiments of the invention will now be demonstrated by reference to the following examples. It should be understood that these examples are disclosed solely by way of illustrating the invention and should not be taken in any way to limit the scope of the present invention.

A shell composition according to an embodiment of the present disclosure was prepared. The shell composition is presented in Table 1.

TABLE 1
Shell composition including Amidated Pectin

| Ingredients | % |
|---|---|
| Gelatin | 25-55 |
| Glycerin | 8-35 |
| Pectin | 3-22 |
| Dextrose | 0.01-1.0 |
| Gellan Gum | 0.2-8 |
| Purified Water | 8-20 |
| Total | 100.0 |

Batch manufacturing of a softgel capsule according to an embodiment of the present disclosure was performed. The shell composition of Table 1 was prepared using a Ross melter. Valproic acid was used as the active pharmaceutical ingredient. The valproic acid was dispensed into a stainless steel hopper under nitrogen blanketing. A rotary die encapsulation machine was used to encapsulate the valproic acid in the shell composition.

The softgel capsules were dried using a drying chamber. After drying was completed, the softgel capsules were inspected for defects and washed using a lecithin/ethanol solution.

A stability study was performed on the softgel capsules. The softgel capsules were packaged into 200 cc HDPE bottles and was put under various stability conditions, 25° C./60% RH, 30° C./65% RH and 40° C./75% RH.

The softgel capsules were subjected to a two-stage dissolution to evaluate its their enteric property. USP <711> was followed. USP APP II with 50 RPM paddle speed was used. The softgel capsules were expected to be intact for two hours in 0.1N HCl and ruptured within 45 minutes in pH 6.8 phosphate buffer. The results of the two stage dissolution test are presented in Table 2.

TABLE 2
Two Stage Dissolution Test Results (USP APP II, 50 RPM Paddle Speed)

| | | | T = 3 Months | | T = 6 Months | | T = 27 Months | |
|---|---|---|---|---|---|---|---|---|
| | T = 0 | | 40° C./75% RH | | 40° C./75% RH | | 25° C./60% RH | |
| | Acid | Buffer | Acid | Buffer | Acid | Buffer | Acid | Buffer |
| | Stage | Stage | Stage | Stage | Stage | Stage | Stage | Stage |
| Lot No. | (0.1N HCl) | (pH 6.8) | (0.1N HCl) | (pH 6.8) | (0.1N HCl) | (pH 6.8) | (0.1N HCl) | (pH 6.8) |
| 18MC- | Intact for | 9 min | Intact for | 12 min | Intact for | 11 min | Intact for | 7 min |
| 86 | 120 min | | 120 min | | 120 min | | 120 min | |

From these results, it was found that the softgel capsules were stable over a period of 6 months at 40° C./75% RH condition and 27 months at 25° C./60% RH condition.

Analytical testing for impurities was also performed on the samples stored at 25 C/60% RH condition for 27 months. The test method was per current USP Monograph for valproic acid. The testing results are summarized in Table 3.

TABLE 3
Impurity Test Results for T = 27 Months at 25° C./60% RH

| Impurity | % | Specification |
|---|---|---|
| RRT ~0.152 | 0.01% | Individual Impurities: |
| Butyric Acid | ND^{#} | NMT 0.1% |
| Valeric Acid | 0.01% | Total Impurities: |
| RRT ~0.545 | 0.02% | NMT 0.3% |
| RRT ~0.873 | 0.01% | |
| RRT ~1.397 | 0.01% | |
| Related Compound A | 0.05% | |
| Total Impurities | 0.11% | |

^{#}Not detected.

The testing results with total impurities of 0.11% after 27 months at ambient condition demonstrated that the valproic acid softgel product is chemically stable. From these results, the softgel capsule according to one embodiment of the present disclosure is a stable product with robust enteric property and chemical stability.

1. A softgel capsule comprising:
(a) a fill material; and
(b) a shell composition,
wherein the fill material comprises valproic acid or a salt thereof, and
wherein the shell composition comprises a gelatin, a plasticizer, and an enteric polymer.

2. The softgel capsule of claim 1, wherein the shell composition further comprises dextrose.

3. The softgel capsule of claim 1, wherein the enteric polymer is pectin.

4. The softgel capsule of claim 3, wherein the pectin is an amidated pectin, a non-amidated pectin or combinations thereof.

5. The softgel capsule of claim 1, wherein the shell composition comprises about 25 wt % to about 55 wt % of a gelatin.

6. The softgel capsule of claim 1, wherein the shell composition comprises about 3 wt % to about 22 wt % of pectin.

7. The softgel capsule of claim 1, wherein the shell composition comprises about 0.01 wt % to about 1.0 wt % of dextrose.

8.-10. (canceled)

11. The softgel capsule of claim 1, wherein the plasticizer is glycerin, aqueous sorbitol and sorbitan solution or combinations thereof.

12. (canceled)

13. (canceled)

14. The softgel capsule of claim 1, wherein the shell composition further comprises a gelling agent.

15. (canceled)

16. (canceled)

17. The softgel capsule of claim 1, wherein the fill composition further comprises an antioxidant.

18. The softgel capsule of claim 17, wherein the antioxidant comprises butylated hydroxytoulene (BHT) or butylated hydroxyanisole (BHA).

19. (canceled)

20. The softgel capsule of claim 1, wherein the fill composition comprises from about 125 mg to about 1000 mg of valproic acid.

21. (canceled)

22. (canceled)

23. The softgel capsule of claim 1, wherein the softgel capsule has a stability of at least about 90% when tested at 25° C./60% RH at 1 month, 2 months, 3 months, 6 months, 12 months, or 24 months.

24.-28. (canceled)

29. The softgel capsule of claim 1, wherein the softgel capsule has a stability of at least about 90% when tested at 30° C./65% RH at 1 month, 2 months, 3 months, 6 months, or 12 months.

30.-34. (canceled)

35. The softgel capsule of claim 1, wherein the softgel capsule has a stability of at least about 90% when tested at 40° C./75% RH at 1 month, 2 months, 3 months, or 6 months.

36-45. (canceled)

46. The softgel capsule of claim 1, wherein the total impurity does not exceed about 0.5%, about 0.4%, about 0.3%, about 0.2%, about 0.1% or about 0.01%, wherein the total impurity is measured per current USP Monograph test method.

47. (canceled)

48. A process for producing the softgel capsule of claim 1, comprising encapsulating the fill composition in the shell composition to form the softgel capsule and drying the softgel capsule.

49. The process of claim 48, wherein prior to encapsulating a gel conversion occurs for the shell composition.

50. A method of treating seizures comprising administering a softgel capsule of claim 1.

51. A method of treating bipolar disorder comprising administering a softgel capsule of claim 1.

52. (canceled)
